(19) United States
(12) Patent Application Publication
(10) Pub. No.: US 2008/0286337 A1
(43) Pub. Date: Nov. 20, 2008

(54) Method of treating a disease in an eye using a scleral lens

(75) Inventors: Perry Rosenthal, Newton, MA (US); Deborah S. Jacobs, Brookline, MA (US)

Correspondence Address: WILMERHALE/BOSTON, 60 STATE STREET, BOSTON, MA, 02109, US

(73) Assignee: Boston Foundation for Sight, Needham, MA (US)

(21) Appl. No.: 11/803,709

(22) Filed: May 15, 2007

(51) Int. Cl. A61F 2/14; A61K 31/56; A61K 38/16; A61K 39/395; A61K 9/00; A61P 27/02

(52) U.S. Cl. 424/427; 424/130.1; 424/145.1; 424/158.1; 514/12; 514/178

(57) ABSTRACT

A scleral lens is provided with a drug that is retained in the reservoir of fluid between the scleral lens and the cornea. This system can be used to deliver drugs not currently used because of poor bioavailability, to increase bioavailability of drugs used in patients already wearing a scleral lens, and to improve bioavailability in patients who are not currently wearing the lens. Dosing can be provided less frequently, thus decreasing the risk of non-compliance.

### TECHNICAL FIELD

This disclosure relates generally to retaining a drug at the surface of an eye when a scleral lens is disposed on the eye, retaining stem cells in an aqueous environment at the surface of the eye when a scleral lens is disposed on the eye, a method of using a scleral lens to retain a drug at the surface of the eye, a method of tissue engineering using a scleral lens to retain stem cells in an aqueous environment when delivered to the surface of the eye, and methods for treating conditions and diseases of the eye.

### BACKGROUND

The eye's most important focusing lens is the cornea, which is the transparent dome-shaped front part of the eye. The cornea should have a perfectly smooth surface in order to provide clear vision. When disease or injury causes irregularity to the corneal surface the eye can no longer focus clearly, even with the strongest glasses. Hard corneal contact lenses can improve the vision of irregular corneas by creating a smooth layer of tears that covers the irregularities. However, many eyes with damaged corneas cannot be fitted with a hard corneal contact lens. Moreover, the corneas of patients who suffer from severe ocular surface disease can become too fragile to withstand exposure to air or the pressure of a blink, and even less so the friction of a hard corneal contact lens.

It is known that eye diseases can be treated with topical agents, such as drops or ointments, which use direct absorption to reach therapeutic drug levels at the target tissue. Topical application is especially useful for the cornea, which has virtually no blood supply and is easily accessible for topical application. When drugs are applied topically in the form of drops or ointment to the cornea, however, the drug solution rapidly disperses into the tear film and flows into the tear drainage system, thereby reducing bioavailability. Non-limiting examples of factors affecting the bioavailability of a drug (i.e., drug levels) in the cornea, and to the anterior chamber of the eye, include how long the drug is present in the tear film, its absorption from the tear film into the corneal tissue, and the frequency of application. Dosing regimens of four times per day are typical; hourly dosing is not unusual in sight-threatening conditions.

An existing method of delivering a drug to the cornea involves dehydrating a soft contact lens, then soaking the lens in a solution of the drug. This method provides an initial burst release of the drug followed by continuous decline in the amount of drug at the cornea. This leads to limited bioavailability of the drug after the initial burst release.

Another method of delivering a drug to the cornea involves soaking a collagen shield in a solution of the drug. These collagen shields are opaque, so the patient cannot see while wearing them. This method also has a burst release, followed by limited bioavailability.

A scleral lens is described in U.S. application Ser. No. 11/473,290 (published as US 2006/0290883), which is incorporated herein by reference in its entirety. A method of making such a scleral lens is described in U.S. Pat. No. 5,452,031, which is incorporated herein by reference in its entirety.

### SUMMARY

A scleral lens rests on the sclera and creates a vaulted area over the cornea, which defines a reservoir of fluid between the inner surface of the vaulted area and the cornea. This reservoir of fluid, which is referred to as an expanded pre-corneal tear film or as a supplemented pre-corneal tear film, acts as a “liquid bandage.” A scleral lens allows for improved retention of a drug at the surface of the eye in this expanded pre-corneal tear film. Alternatively, a scleral lens may be used to retain stem cells in an aqueous environment at the surface of the eye.

The scleral lens also provides a novel method of retaining a drug at the corneal surface in the expanded pre-corneal tear film, which is retained at the surface of the cornea by the scleral lens. The drug may be added to the scleral lens, which is then inserted into the eye. Alternatively, the drug is administered to the eye, then the scleral lens is inserted into the eye. Stem cells may be used instead of a drug.

A scleral lens provides high bioavailability of drugs to the relatively avascular cornea, and perhaps to the anterior chamber of the eye, without the potential risks of systemic administration or the requirement of frequent administration. The scleral lens retains the drug at the cornea, thus maintaining a high concentration of the drug at the site of administration compared to other known methods. This requires less frequent dosing regimens, which in turn reduces noncompliance. The scleral lens provides these benefits while keeping the cornea oxygenated and allowing the patient to see, or even enhancing the patient's vision.

Non-limiting examples of uses for the scleral lens drug delivery system include drugs that are not currently administered topically to the eye because of poor bioavailability, to improve bioavailability of drugs used in patients already using a scleral lens, and to improve bioavailability in patients who are not otherwise in need of a scleral lens.

The scleral lens drug delivery system can also be used to maintain an aqueous environment for stem cells delivered to the eye, and to help retain stem cells at the surface of the cornea. Non-limiting examples of diseases that may be treated using the present invention with stem cells include chemical burns, Stevens-Johnson Syndrome, aniridia, and ocular cicatricial pemphigoid. It is known that limbal stem cells may be transplanted to an eye that has suffered damage to the epithelial layer of the cornea. These transplanted limbal stem cells will produce a new healthy layer of epithelial cells in the damaged eye. Non-limiting examples of stem cell delivery configurations useful with the present invention include sheets of stem cells and stem cells in solution.

The invention relates to the realization that a lens disposed on an eye with a drug may be used to treat a disease in the eye. Accordingly, the invention relates to a method of treating a disease in the eye of a patient by administering to the eye a pharmaceutically effective amount of a drug and inserting a lens having a back surface comprising an optic portion and a scleral portion (haptic), the haptic portion having an outer rim and an inner rim. In one embodiment of the invention, the lens is a scleral lens. In another embodiment of the invention, the lens is a scleral lens that contacts only the sclera. In another embodiment of the invention, the lens is a scleral lens that contacts only the sclera and the periphery of the cornea. In yet another embodiment of the invention, the lens is a scleral lens and the haptic portion of the scleral lens further defines a channel that extends radially at least part of the distance between the outer rim and the inner rim. In some cases, the patient of the invention is in need of such treatment.

In one embodiment of the invention, the drug is an antibiotic, an antiviral, an antifungal, an antiparasitic, a corticosteroid, a non-steroidal anti-inflammatory, a mydriatic, a biologic, a drug that modifies neovascularization, a drug that increases aqueous outflow, a drug that reduces aqueous secretion, an antihistamine, a secretagogue, a mast cell stabilizer, a tear supplement, an anti-metabolite, or an immunomodulator. In another embodiment of the invention, the drug is a protein. In yet another embodiment, the drug of the invention is an antibody or an antibody fragment. In some cases, the drug of the invention in an antibody or antibody fragment that interacts with a vascular endothelial growth factor or a vascular endothelial growth factor receptor. In yet another embodiment, the drug of the invention is bevacizumab, pegaptanib, or ranibizumab.

In one embodiment of the invention, the disease is bacterial infection, viral infection, fungal infection, parasitic infection, inflammation, neovascularization, ocular surface disease, glaucoma, allergy, dry eye, dysplasia, and neoplasm.

In one embodiment of the invention, the lens is an optically corrective lens. In another embodiment of the invention, the lens is not an optically corrective lens.

In one embodiment of the invention, the drug is added to the lens, then the lens is inserted into the eye. In another embodiment of the invention, the drug is added to the eye, then the lens is inserted into the eye.

### BRIEF DESCRIPTION OF THE DRAWINGS

In the Drawings:

FIG. 1 is a perspective view of an eye with a scleral lens;

FIG. 2 is a sectional view taken from lines 2-2 in FIG. 1;

FIG. 3 is a top view of a scleral lens;

FIG. 4 is a detail view of a particular embodiment of a scleral lens;

FIG. 5 is a composite of clinical photos demonstrating regression of corneal neovascularization using the scleral lens drug delivery system for the drug AVASTIN® (bevacizumab)

### DETAILED DESCRIPTION

The present inventions provide a scleral lens and a drug which is retained in the expanded pre-corneal tear film defined as the area between the scleral lens and the cornea. In another embodiment, the present inventions provide a scleral lens and stem cells in the aqueous environment defined as the area between the scleral lens and the cornea. The scleral lens is made of a rigid gas permeable material. Additionally, the scleral lens may have channels which are disposed on the inside surface of the scleral lens and extend generally radially from the inside of the scleral contact portion (haptic) of the scleral lens and the outside rim of the scleral lens.

The present inventions also provide a method of increasing the bioavailability of a drug delivered to the surface of the eye using a scleral lens, whereby a solution of a drug is added to the expanded pre-corneal tear film defined as the area between the scleral lens and the cornea. In another embodiment, the present inventions provide a method of retaining stem cells at the surface of the eye in an aqueous environment using a scleral lens.

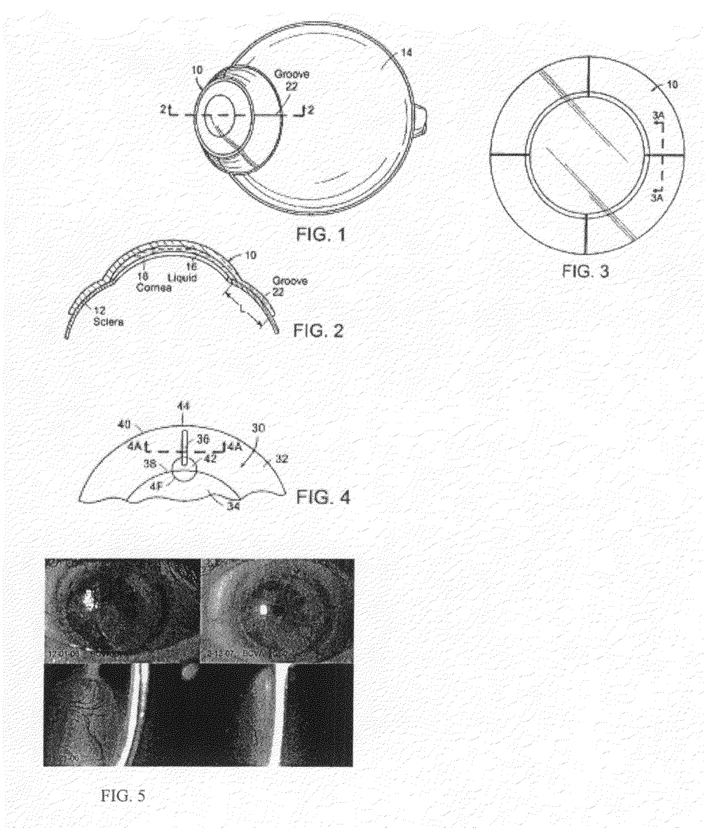

A scleral lens is a device that can be used to address corneal surface conditions. As shown in FIGS. 1-4, a scleral lens 10, which is about the size of a quarter, rests on the white sclera 12 of an eye 14. Lens 10 has a dome-shape that defines a fluid compartment space 16 over a cornea 18 that can be filled with fluid, such as artificial tears. As illustrated by the contact length L in FIG. 2, contact with the eye is limited to the sclera and the lens does not contact cornea 18. Alternatively, the scleral lens contacts the sclera and the periphery of the cornea. This contact location for the scleral lens is different from conventional contact lenses which rest on the cornea. By covering (without contacting) the irregular surface of the damaged cornea, this lens device can improve vision in eyes with distorted corneas, and even ones with extreme distortion. The optic portion 34 of the scleral lens may be formed to correct the vision of the patient. Such a scleral lens is an optically corrective lens. Alternatively, the optic portion 34 may not help to correct the vision of the patient.

The fluid compartment becomes a “liquid bandage,” which is referred to as an expanded pre-corneal tear film or as a supplemented pre-corneal tear film, defined by lens 10 that protects the cornea from dryness and from mechanical trauma from the lids with each blink. The expanded pre-corneal tear film is a therapeutic environment that supports healing and that can reduce or even virtually eliminate pain and photosensitivity. This expanded pre-corneal tear film has helped, and even been responsible for the extraordinary healing experienced by, patients who have used the scleral lens. A scleral lens can be used to treat a number of conditions, including dry eyes and keratoconus.

A scleral lens can be used as a drug delivery system by providing a drug in the expanded pre-corneal tear film defined by the inner surface of the scleral lens and the surface of the cornea. The drug is thereby retained in the expanded pre-corneal tear film between the lens and the eye.

The present inventions provide a system for delivering antibiotic agents, antiviral agents, antifungal agents, antiparasitic agents, corticosteroids, non-steroidal anti-inflammatory drugs, mydriatics, biologics, drugs that modify neovascularization, tissue engineering components, drugs that increase aqueous outflow, drugs that reduce aqueous secretion, antihistamines, mast cell stabilizers, secretagogues, tear supplements, anti-metabolites, and immunomodulators to the eye.

The present inventions also provide a method for treating disease processes of the eye, including bacterial infection, viral infection, fungal infection, parasitic infection, inflammation, neovascularization, ocular surface disease, glaucoma, allergy, dry eye, dysplasia, and neoplasm.

Corneal neovascularization (i.e., formation of new blood vessels) is a pathologic process that occurs as part of healing after infection, trauma, or corneal inflammatory processes. Although neovascularization is part of the scar that prevents perforation and loss of the eye, its presence puts the eye at risk of rejection should cornea transplantation be required. Neovascularization can cause calcium and cholesterol deposits and often has associated fibrous tissue, all of which can preclude fine vision. There is little that can be done to treat the eye once neovascularization begins, other than applying a topical steroid (which acts non-specifically) and treating the underlying disease.

Some drugs that block neovascularization interact with Vascular Endothelial Growth Factor (VEGF) or its receptors (VEGFR). Non-limiting examples of VEGF include VEGF-A, VEGF-B, VEGF-C, and VEGF-D. Non-limiting examples of VEGFR include VEGFR-1, VEGFR-2, and VEGFR-3. Non-limiting examples of drugs that block neovascularization by interacting with VEGF or VEGFR include AVASTIN® (bevacizumab), MACUGEN® (pegaptanib sodium injection), and LUCENTIS® (ranibizumab injection).

AVASTIN® (bevacizumab), a recombinant humanized monoclonal IgGl antibody that binds to and inhibits the biologic activity of VEGF, has been approved for the systemic treatment of colon cancer. AVASTIN® is also used off-label as an injection into the eye for retinal neovascularization associated with Age Related Macular Degeneration (AMD). MACUGEN® (pegaptanib sodium injection), an anti-VEGF inhibitor, and LUCENTIS® (ranibizumab injection), an antibody fragment designed to bind and inhibit VEGF-A, are approved for injection into the eye for AMD.

As illustrated in FIG. 4, a portion of a scleral lens 30 includes both a scleral contact surface 32 and a vaulting lens portion 34. The vaulting lens portion is disposed above the cornea when the scleral lens is applied to an eye. The scleral surface of the eye contacts the lens at the scleral contact surface 32. In an alternate embodiment, the scleral lens may rest on the sclera and the periphery of the cornea. As illustrated in FIG. 2, liquid 16 is interposed between the inner surface of the lens portion 10 and the surface of the cornea 18. Drug is contained in this liquid, thus keeping the drug in contact with the surface of the cornea. In another embodiment, the liquid contains stem cells and retains them at the surface of the cornea in an aqueous environment.

Drugs suitable for use with the present invention include antibiotic agents, antiviral agents, antifungal agents, antiparasitic agents, corticosteroids, non-steroidal anti-inflammatory drugs, mydriatics, biologics, drugs that modify neovascularization, tissue engineering components, drugs that increase aqueous outflow, drugs that reduce aqueous secretion, antihistamines, mast cell stabilizers, secretagogues, tear supplements, and anti-metabolites.

This drug delivery system can be used to treat disease processes of the eye, including bacterial infection, viral infection, fungal infection, parasitic infection, inflammation, neovascularization, ocular surface disease, glaucoma, allergy, dry eye, dysplasia, and neoplasm.

In another embodiment, the method of the present invention is performed by adding a solution containing stem cells to the expanded pre-corneal tear film. Non-limiting examples of diseases that may be treated using the method of the present invention with stem cells include chemical burns, Stevens-Johnson Syndrome, aniridia, and ocular cicatricial pemphigoid.

### Drugs Useful in the Invention

Non-limiting examples of drugs suitable for use with the present inventions include antibiotic agents, antiviral agents, antifungal agents, antiparasitic agents, corticosteroids, non-steroidal anti-inflammatory drugs, mydriatics, biologics, drugs that modify neovascularization, tissue engineering components, drugs that increase aqueous outflow, drugs that reduce aqueous secretion, antihistamines, mast cell stabilizers, secretagogues, tear supplements, anti-metabolites, and immunomodulators.

The term “antibiotic” as used herein is defined as a drug that kills or prevents the growth of bacteria. Non-limiting examples of antibiotics useful in the present inventions include POLYTRIM® (trimethoprim sulfate/polymyxin B sulfate), ZYMAR® (gatifloxacin), and VIGAMOX® (moxifloxacin hydrochloride).

The term “antiviral” as used herein is defined as a drug that treats viral infections. A non-limiting example of an antiviral useful in the present inventions is VIROPTIC® (trifluridine).

The term “antifungal” as used herein is defined as a drug that prevents the growth of fungi. A non-limiting example of an antifungal useful in the present inventions is Natamycin (pimaricin).

The term “antiparasitic” as used herein is defined as a drug that treats infection by parasites. Non-limiting examples of antiparasitics useful in the present inventions are Brolene (propamidine isethionate), HIBICLENS® (chlorhexidene gluconate), and PERIOSTAT® (doxycycline hyclate).

The term “corticosteroid” as used herein is defined as a class of steroid hormones that are useful for regulating physiologic systems such as stress response, immune response and regulation of inflammation, carbohydrate metabolism, protein catabolism, blood electrolyte levels, and behavior. Non-limiting examples of corticosteroids useful in the present inventions include PRED FORTE® (prednisolone acetate), LOTEMAX® (loteprednol etabonate), and FML FORTE® (fluorometholone)

The term “non-steroidal anti-inflammatories” as used herein is defined as drugs with analgesic, antipyretic and anti-inflammatory effects that are non-steroidal. Non-limiting examples of non-steroidal anti-inflammatories useful in the present inventions include ACULAR® (ketorolac tromethamine) and VOLTAREN® (diclofenac).

The term “mydriatic” as used herein is defined as a drug that induces dilation of the pupil. A non-limiting example of a mydriatic useful in the present inventions is ISOPTO® HYOSCINE (scopolamine).

The term “biologic” as used herein is defined as a product that may be composed of sugars, proteins, or nucleic acids or complex combinations of these substances, or may be living entities such as cells and tissues. Non-limiting examples of biologics useful in the present inventions include AVASTIN® (bevacizumab), MACUGEN® (pegaptanib), LUCENTIS® (ranibizumab), autologous serum, fetal cord serum, and amniotic membrane extracts.

The term “drug that modifies neovascularization” as used herein is defined as a drug that modifies the formation of new blood vessels. Non-limiting examples of drugs that modify neovascularization useful in the present inventions include PRED FORTE® (prednisolone acetate), AVASTIN® (bevacizumab), MACUGEN® (pegaptanib), and LUCENTIS® (ranibizumab).

The term “tissue engineering component” as used herein is defined as a material used to repair or replace portions of tissues or whole tissues. Non-limiting examples of tissue engineering components useful in the present inventions include limbal stem cells, autologous stem cells, and allogeneic stem cells.

The term “drug that increases aqueous outflow” as used herein is defined as a drug that increases pressure in the eye by increasing the production of aqueous. Non-limiting examples of drugs that increase aqueous outflow useful in the present inventions include pilocarpine, XALATAN® (latanoprost), TIMOPTIC® (timolol), and ALPHAGAN® (brimonidine).

The term “drug that reduces aqueous secretion” as used herein is defined as a drug that decreases pressure in the eye by reducing the production of aqueous. Non-limiting examples of drugs that reduce aqueous outflow useful in the present inventions include TRUSOPT® (dorzolamide), AZOPT® (brinzolamide), TIMOPTIC® (timolol), and ALPHAGAN® (brimonidine).

The term “antihistamine” as used herein is defined as a drug that reduces or that eliminates the effects of histamine. Non-limiting examples of antihistamines useful in the present inventions include PATANOL® (olopatadine), ELESTAT® (epinastine), and ZADITOR® (ketotifen fumarate).

The term “mast cell stabilizer” as used herein is defined as a cromone medication that prevents or controls allergic disorders by preventing the release of histamine. Non-limiting examples of mast cell stabilizers useful in the present inventions include ALOMIDE® (lodoxamide), PATANOL® (olopatadine), and ELESTAT® (epinastine).

The term “secretagogue” as used herein is defined as a substance that causes other substances, such as tears, to be secreted. A non-limiting example of a secretagogue useful in the present inventions is PROLACRIA® (diquafosol tetrasodium).

The term “tear supplement” as used herein is defined as a fluid used to increase wetness of the eye. Non-limiting examples of tear supplements useful in the present inventions include REFRESH DRY EYE THERAPY®, REFRESH TEARS®, GENTEAL®, THERATEARS®, and BIONTEARS®.

The term “anti-metabolite” as used herein is defined as a structural analog of a naturally occurring compound, and that interferes with the production of nucleic acids. Non-limiting examples of anti-metabolites useful in the present inventions include mitomycin C and 5-fluorouracil.

The term “immunomodulator” as used herein is defined as an agent that specifically or nonspecifically augments or diminishes immune responses. A non-limiting example of a immunomodulator useful in the present inventions is RESTASIS® (cyclosporine).

### EXAMPLE 1

AVASTIN® is not believed to effectively treat the cornea because the molecule is large and is therefore poorly absorbed into the cornea. A recent report using AVASTIN®, off-label, topically, for the treatment of corneal neovascularization rebuts this perception (Terry Kim, M.D., Cornea Society November 2006, in press Arch. Ophthalmology April 2007). This study required a high concentration of the preservative BAK in the AVASTIN® delivery vehicle to aid absorption of the drug. However, BAK can be toxic to the corneal epithelium. Therapeutic effect was reported after a solution of 1% AVASTIN® and 0.01% BAK was used, one drop, four times per day for at least thirty days.

This study using AVASTIN® off-label, topically, to treat corneal neovascularization was repeated using the scleral lens drug delivery system. One drop of 1% AVASTIN® was added to the expanded pre-corneal tear film of artificial tears in a scleral lens twice per day, and achieved a therapeutic effect by thirty days with continued benefit over the subsequent sixty days. This therapeutic effect was achieved without BAK. Instead, the expanded pre-corneal tear film of the scleral lens drug delivery system provided adequate bioavailability of the drug without exposing the cornea to BAK. FIG. 5 is a composite of clinical photos demonstrating regression of corneal neovascularization using the scleral lens drug delivery system for AVASTIN®.

1. A method of treating a disease in the eye of a patient, the method comprising:
administering to the eye a pharmaceutically effective amount of a drug and
inserting a lens having a back surface comprising an optic portion and a scleral portion (haptic), the haptic portion having an outer rim and an inner rim.

2. The method of claim 1, wherein the lens is a scleral lens.

3. The method of claim 2, wherein the scleral lens contacts only the sclera.

4. The method of claim 2, wherein the scleral lens contacts only the sclera and the periphery of the cornea.

5. The method of claim 2, wherein the haptic portion of the scleral lens further defines a channel that extends radially at least part of the distance between the outer rim and the inner rim.

6. The method of claim 1, wherein the patient is in need of such treatment.

7. The method of claim 1, wherein the drug is selected from the group consisting of an antibiotic, an antiviral, an antifungal, an antiparasitic, a corticosteroid, a non-steroidal anti-inflammatory, a mydriatic, a biologic, a drug that modifies neovascularization, a drug that increases aqueous outflow, a drug that reduces aqueous secretion, an antihistamine, a secretagogue, a mast cell stabilizer, a tear supplement, an anti-metabolite, and an immunomodulator.

8. The method of claim 7, wherein the drug is a biologic.

9. The method of claim 8, wherein the drug is a protein.

10. The method of claim 9, wherein the drug is an antibody or an antibody fragment.

11. The method of claim 10, wherein the antibody or antibody fragment interacts with a vascular endothelial growth factor or a vascular endothelial growth factor receptor.

12. The method of claim 11, wherein the antibody or antibody fragment is selected from the group consisting of bevacizumab, pegaptanib, and ranibizumab.

13. The method of claim 7, wherein the drug is an antibiotic.

14. The method of claim 7, wherein the drug is an antiviral.

15. The method of claim 7, wherein the drug is an antifungal.

16. The method of claim 7, wherein the drug is an antiparasitic.

17. The method of claim 7, wherein the drug is a corticosteroid.

18. The method of claim 7, wherein the drug is a non-steroidal anti-inflammatory.

19. The method of claim 7, wherein the drug is a mydriatic.

20. The method of claim 7, wherein the drug is a drug that modifies neovascularization.

21. The method of claim 7, wherein the drug is a drug that increases aqueous outflow.

22. The method of claim 7, wherein the drug is a drug that decreases aqueous secretion.

23. The method of claim 7, wherein the drug is an antihistamine.

24. The method of claim 7, wherein the drug is a secretagogue.

25. The method of claim 7, wherein the drug is a mast cell stabilizer.

26. The method of claim 7, wherein the drug is a tear supplement.

27. The method of claim 7, wherein the drug is an anti-metabolite.

28. The method of claim 7, wherein the drug is an immunomodulator.

29. The method of claim 5, wherein the disease is selected from the group consisting of bacterial infection, viral infection, fungal infection, parasitic infection, inflammation, neovascularization, ocular surface disease, glaucoma, allergy, dry eye, dysplasia, and neoplasm.

30. The method of claim 29, wherein the disease is bacterial infection.

31. The method of claim 29, wherein the disease is viral infection.

32. The method of claim 29, wherein the disease is fungal infection.

33. The method of claim 29, wherein the disease is parasitic infection.

34. The method of claim 29, wherein the disease is inflammation.

35. The method of claim 29, wherein the disease is neovascularization.

36. The method of claim 29, wherein the disease is ocular surface disease.

37. The method of claim 29, wherein the disease is glaucoma.

38. The method of claim 29., wherein the disease is allergy.

39. The method of claim 29, wherein the disease is dry eye.

40. The method of claim 29, wherein the disease is dysplasia.

41. The method of claim 29, wherein the disease is neoplasm.

42. The method of claim 1, wherein the lens is an optically corrective lens.

43. The method of claim 1, wherein the lens is not an optically corrective lens.

44. The method of claim 1, wherein the drug is added to the lens, then the lens is inserted into the eye.

45. The method of claim 1, wherein the drug is added to the eye, then the lens is inserted into the eye.
